(19) United States
(12) Patent Application Publication
(10) Pub. No.: US 2018/0184915 A1
(43) Pub. Date: Jul. 5, 2018

(54) Bio-Sensing Glasses to Detect Cardiovascular Disease

(71) Applicant: AWAIRE INC., San Jose, CA (US)

(72) Inventors: Anurag Darbari, San Jose, CA (US); Archana Darbari, San Jose, CA (US)

(21) Appl. No.: 15/684,615

(22) Filed: Aug. 23, 2017

Related U.S. Application Data
(60) Provisional application No. 62/442,381, filed on Jan. 4, 2017.

(51) Int. Cl. A61B 5/0205; A61B 5/00; A61B 7/04; A61B 5/02; A61B 7/00; A61B 5/1486; A61B 5/145

(52) U.S. Cl. CPC A61B 5/02055; A61B 5/6803; A61B 7/04; A61B 5/02007; A61B 7/003; A61B 5/021; A61B 5/14517; A61B 5/14532; A61B 5/14546; A61B 5/7264; A61B 5/7275; A61B 5/1486

(57) ABSTRACT

Bio-sensing devices are embedded as part of smart eye-wear system, comprising of a pair of temples and a nose pad. A plurality of sensors disposed within the eye-glasses and are adapted to acquire, analyze, and classify biometric information to monitor and detect abnormal heart behavior and to predict congestive heart failure. Using Machine Learning methods to monitor and detect abnormal heart behavior and classify/predict congestive heart failure, depends on sensing device to capture and provide bio-signals, to provide meaningful and usable extracted features by the machine. Sensing Device, capable of capture meaningful and useful bio-signals generated by the human body is discussed, which are “pressure wave sensor”, capable of acquiring acoustic pressure wave signal generated by the body and extracting heart sound from the sensor. Additionally, ion exchange sensing device, which is capable of extracting sodium, potassium, glucose and lactate information from human sweat is also discussed.

### CROSS-REFERENCE TO RELATED APPLICATION(S)

The present application claims priority to U.S. Provisional Patent Application No. 62/442,381 filed on Jan. 4, 2017, entitled “Bio-Sensing Glasses to detect Cardiovascular Disease” the entire disclosure of which is incorporated by reference herein.

### BACKGROUND OF THE INVENTION

### 1. Field of Invention

The present invention relates to the field of wearable bio-sensing devices, and more specifically to the field of biosensors for cardiovascular disease.

### 2. Description of Related Art

Commercially available wearable sensors have limited capabilities, eg. tracking individual's physical activities and vital signs, such as temperature, heart rate, steps climbed, and do not provide insight into user's health at molecular levels. Wearable sensor technology is driving personal health care closer in providing health analysis, by continuously monitoring various health states.

Cardiovascular related deaths are the primary cause of deaths worldwide, with approximately 18 million deaths occurring annually. At this time, there is no known solution that addresses this problem by effectively reduce the number of cardiovascular deaths.

Solution to above mentioned problem:
a. Heart acoustic sound when combined with body sweat provides vital information about heart health, providing indication about possible issues with the hearts structural problems, diseased vessels, and/or blood clots. Heart sound analysis detecting abnormalities in acquired sound, is done by using machine learning techniques.
b. Human sweat provides insight regarding body vitals at the molecular level by measuring the level of sodium, potassium, and glucose level generated by human body. Information extracted from human sweat containing physiologically and metabolically rich information is used to analyze heart health, diabetes detection, disease diagnosis, drug abuse detection, and athletic cardiovascular optimization.
c. Our Invention is a fully integrated ultra-low power-sensing device, which is designed to capture and analyze acoustic sound generated by heart and body sweat generated from body.
d. Using the acoustic sensing device, along with a mobile device application, the consumer can track rhythm disorders, heart rate variability, and also detect numerous heart diseases. Using a bio fluid sensing device, along with the mobile device application, the consumer can track body enzyme and electrolyte level and how it is affecting heart health.
e. These sensing devices are non-invasive, and require no medical assistance during use.

Wearable electronic sensing devices are designed to be worn all day and be in physical contact with human skin. This allows for continuous and close monitoring of an individual's activities without interrupting or limiting the consumer's motions, while providing real-time continuous monitoring of an individual's physiological biomarkers. Potential applications for these sensors optimal use are wearable glasses, head bands, smart watches, necklaces and other wearable devices known in the art.

Based on the foregoing, there is a need in the art for a wearable bio-sensing device that can intake and analyze a plurality of stimuli to convey results to a user.

### SUMMARY OF THE INVENTION

A bio-sensing device comprises a pair of eye-glasses having a pair of temples and a nose pad. A plurality of sensors is disposed within the eye-glasses. Each sensor is adapted to acquire, analyze, and classify biometric information.

In an embodiment, biometric information is selected from a group consisting of pressure, acoustics, temperature, glucose, lactate, potassium ions, and sodium ion sensing elements. The acoustics sensor is adapted to receive biometric acoustic signal, which comprises of heart sound, to classify murmurs, rhythm disorder detection, heart rate variability, stenosis, regurgitation, atrial septal defect, and ventral septal defect.

In an embodiment, at least one of the plurality of sensors is one or more pressure sensors. At least one of the pressure sensors is adapted to receive a heart pressure wave signal, and one of the pressure wave sensors is adapted to receive lung sound.

In an embodiment, at least two of the sensors are electrodes, wherein one is an enzyme sensor and one is an electrolyte sensor. Each electrode receives the biometic information from human sweat. In an embodiment, the information received from the human sweat is glucose, lactate, potassium ions, and sodium ions.

In an embodiment, the pressure wave sensor is positioned on the nose pad and the at least one enzyme sensor is positioned on the pair of temples.

In an embodiment, the device may further comprise accelerometers, gyroscopes, compasses, and altimeters, each of which determines user behavior and activity while other measurements are recorded.

A method for monitoring congestive heart failure comprises the steps of capturing pressure waves using one or more pressure wave sensors, filtering the pressure waves using one or more fixed function hardware accelerators, collecting the pressure waves over an n-cardiac cycles, extracting one or more features using a digital signal processor, and providing extracted features to one or more classifiers. Each classifier is built using a neural network which provides one or more output classification. Each classification determines the presence of one or more abnormal heart sounds.

In an embodiment, abnormal heart conditions are classified by the presence of a murmur, presence of S3/S4 components, click, prolapse, regurgitation and stenosis.

In an embodiment, the step of one or more fixed function hardware accelerators is filtering the pressure waves to isolate heart pressure waves, and filtering one or more autoencoder reconstruct corrupted sections of heart sounds.

In an embodiment, the one or more time domain features are defined, by total power and peak amplitude during the systolic period, total power and peak amplitude during the diastolic period, a zero crossing rate, time duration of one or more S1 heart sounds, time duration of one or more S2 heart sounds, time duration from the end of an S1 to the start of an S2 in one of the n number of cardiac cycles, and time duration of the end of an S2 to the start of an S1 in one of the n number of cycles. The one or more frequency domain features are defined by one or more systolic periods. The one or more statistical domain features are defined by mean, standard deviation, variance, skewness, kurtonsis, sample entropy, and shannon entropy. Cepstrum domain features are defined by cepstrum S1 peak amplitude, S1 peak quefrency, cepstrum S2 peak amplitude, and S2 peak quefrency.

In an embodiment, filtering is facilitated by an environmental hardware accelerator that filters pressure waves from a user's environment.

In an embodiment, a plurality of networks to classify abnormal heart sounds data. A final output classification is provided by the one or more classifiers. Each classifier uses a plurality of algorithms including artificial bee colony and k-nearest neighbor in order to classify congestive heart failure. Congestive heart failure may be classified as at-risk, high risk, developed risk, and require intervention. Classification Result is reinforced using two or more electrode sensors. At least one electrode sensor is an enzyme sensor, lactate levels reinforce the classification, and at least one electrode sensor is an electrolyte sensor, wherein sodium and potassium levels reinforces classification results.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a perspective view of the bio-sensing glasses, according to an embodiment of the present invention;

FIG. 2 is a perspective view of a user wearing the bio-sensing glasses, according to an embodiment of the present invention;

FIG. 3 is a diagram of the nose pad construction, according to an embodiment of the present invention;

FIG. 4 is a side elevation view of the enzyme and electrolyte sensors on the temple, according to an embodiment of the present invention;

FIG. 5 is a diagram of the embedded sensor subsystem, according to an embodiment of the present invention;

FIG. 6 is a diagram representing Analog Front End for the Sensing Devices, according to an embodiment of the present invention;

FIG. 7 is a cardiac acoustic output, at the nose pad, depicting cyclic detection and windowing of Systole and Diastole, according to an embodiment of the present invention;

FIG. 8 is a block diagram which describes classification mechanism deployed to perform classification of heart disease or heart failure, according to an embodiment of the present invention;

FIG. 9 is a diagram view of the heart disease detection process utilizing machine learning, according to an embodiment of the present invention;

FIG. 10 is a diagram of the data flow to detect congestive heart failure, according to an embodiment of the present invention;

FIG. 11 is a block diagram of the fixed function heart activity detector, according to an embodiment of the present invention.

FIG. 12 is a schematic of the network, according to an embodiment of the present invention;

FIG. 13 is a schematic of the data output on a smart device, according to an embodiment of the present invention;

FIG. 14 is a schematic of the data output on a smart device, according to an embodiment of the present invention; and

FIG. 15 is a table of extracted features, according to an embodiment of the present invention.

### DETAILED DESCRIPTION OF PREFERRED EMBODIMENTS

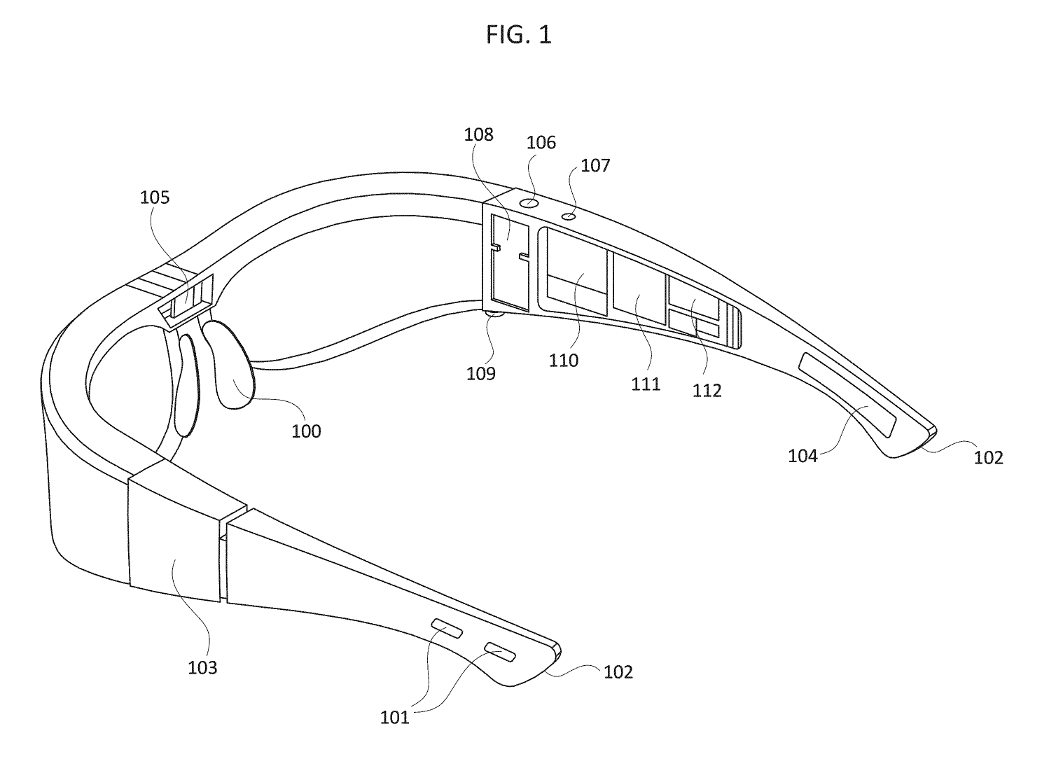

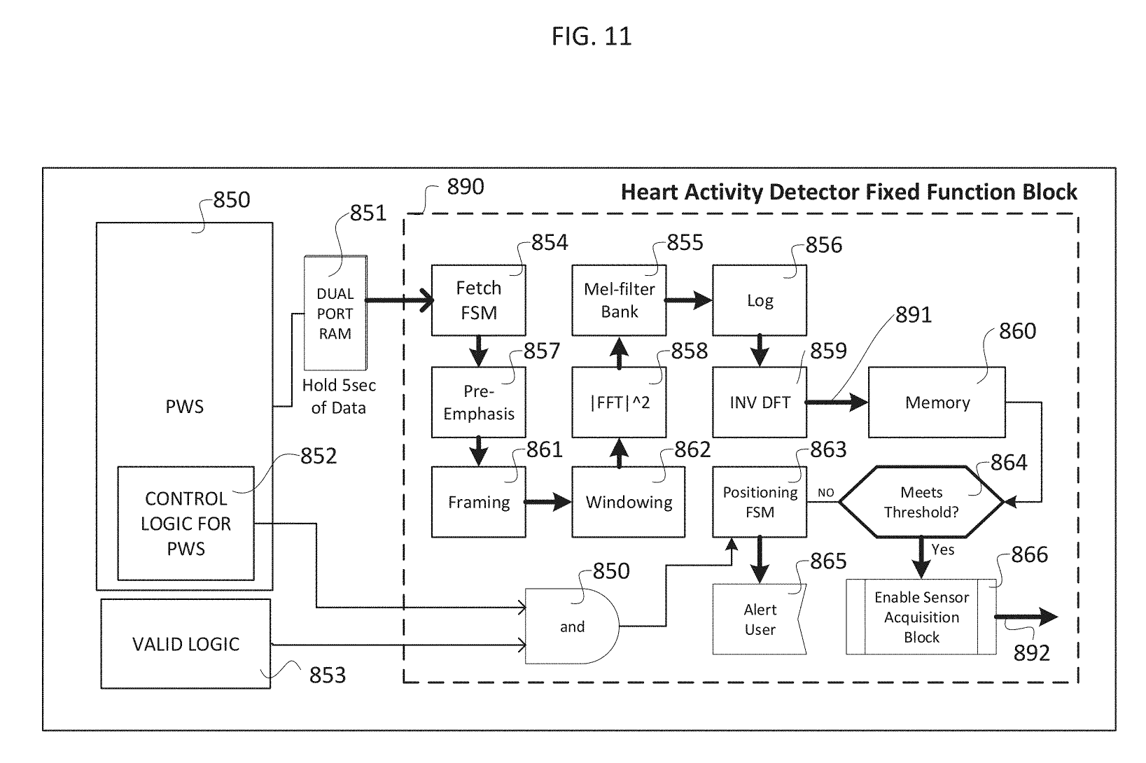

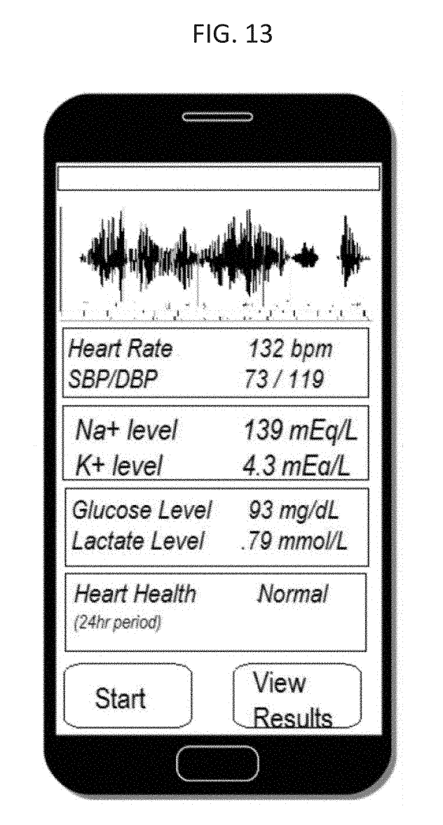

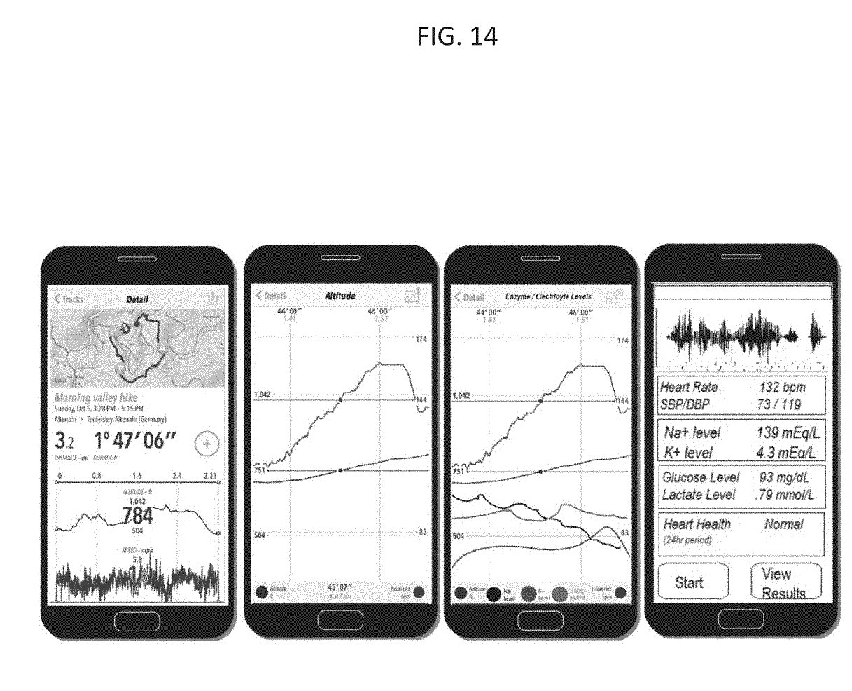

Preferred embodiments of the present invention and their advantages may be understood by referring to FIGS. 1-15, wherein like reference numerals refer to like elements.

The invention disclosed herein relates to the design of sensing elements and their potential application as wearable glasses. These sensing elements are used to analyze heart health, by capturing acoustic sound generated by the heart at the Nasal Bone, as well as body vitals at the molecular level by capturing and analyzing body sweat. The acoustic pressure-sensing element is a solid-state acoustic monitoring device to monitor heart health by capturing and analyzing heart pressure wave signals captured at the nasal bone. A bio-fluid sensing element is solid-state device, designed to capture and analyze electrolyte levels like sodium and potassium, as well as enzymes like glucose and lactate present in human sweat.

The hearts sound wave contains vital information about heart's structural operation and blood flow. Machine analysis provides early indication of abnormal behavior related to the heart.

The human sweat-sensing device provides valuable vital information at the molecular level using an integrated, solid-state, non-invasive, sensing elements. It is important to understand relevant levels of electrolytes and enzymes present in our body such as:
a. Abnormal levels of sodium and potassium (electrolytes) detected in sweat are a potential indicator of a heart problem, and proper level of salts in the body needs to be maintained. High levels of sodium and low level of potassium are especially significant indicators associated with heart problems.
b. Abnormal (high) level of Lactate in Sweat is present when a person is involved with strenuous exercise, or a condition like heart attack, severe infection, lowering overall flow of blood and oxygen throughout the body. Additionally, higher lactate level is recorded again certain medication like metformin (to control diabetes) are consumed.
c. Additionally, as a known fact, patients with type 2 diabetes have increased risk of cardiovascular disease, and it has been concluded that Type 2 diabetic patients with apparently normal cardiac function have impaired myocardial and skeletal muscle energy metabolism related to changes in circulating metabolic substrates. Hence it is important to monitor glucose and lactate level from the body sweat to alert the user, when a similar condition is detected

Using information captured and analyzed from both sensors, a mobile device application monitors heart health; track behavioral habits, along with the body's vitals. In addition, to heart health, the application can also provide insight about potential blood glucose level present in the body.

This invention relates to the design of a non-invasive solid-state sensing device, used to detect heart health by capturing acoustic sound generated by the heart, as well as body vitals at the molecular level present in body sweat. The device operates by capturing acoustic Signal produced by the body, captured at the nasal bone, filtered, and analyzed.

Furthermore, this invention also relates to a sensing device which operates on the bio-fluid obtained from human sweat, generated by body, and selectively screen panel of biomarkers, to compute enzyme and electrolyte levels present in the sweat.

Non-invasive solid-state fully integrated sensing devices are designed to be used in a wearable device, and intended to be in direct contact with the skin to measure biological signals at the molecular level. This device is designed to sense and detect abnormalities and provide early indicator warnings if abnormal heart signal, or abnormal level of electrolytes and metabolites are detected.

Sensing devices are completely integrated unit, includes sensing electrodes, fixed integrated voltage regulators, one or more signal conditioning circuits, pre-processing capabilities, one or more signal processors, and memory

The sensing device is responsible for performing two tasks described herein:
a. First, the device continuously captures acoustic pressure waves generated by the human body in the frequency ranging between infrasound and the audible range. Sensing electrodes capture acoustic pressure signals at the nasal bone, then filters acoustic pressure signals to extract heart signals. Unique feature about this sensing device is that it captures acoustic heart signal at the nasal bone. The device provides continuous filtering of acoustic sound by monitoring validity of heart activity signals, extracting features, generating feature vectors, and computing feature score using support vector machine, linear/logistic regression, kNN, Naive Bayes classifier, and/or back-propagation. The device provides early indication if any anomalies are discovered that could lead to potential structural problem, diseased vessels or blood clots.
b. Second, the device captures bio-fluids in the form of sweat generated by the human body, using solid state enzyme and electrolyte sensing electrodes, sampling human sweat to detect sweat metabolites—glucose/lactate—and electrolytes—sodium/potassium. A unique feature about this sensing device is that it provides vital information at the molecular level using all integrated solid-state non-invasive sweat sensing electrodes. Sweat sensing device operates when electrodes comes in contact with human sweat and generate appropriate electrical signals, indicating levels of electrolytes or enzymes.

These sensing devices are best suited for use in wearable glasses embodiments, however other wearable devices known in the art: such as headbands, smart watches, necklaces, and other wearable devices.

Biosensors are mounted on the nose pad and temple of an eyeglass, to ensure physical contact with the nasal bone and skin.

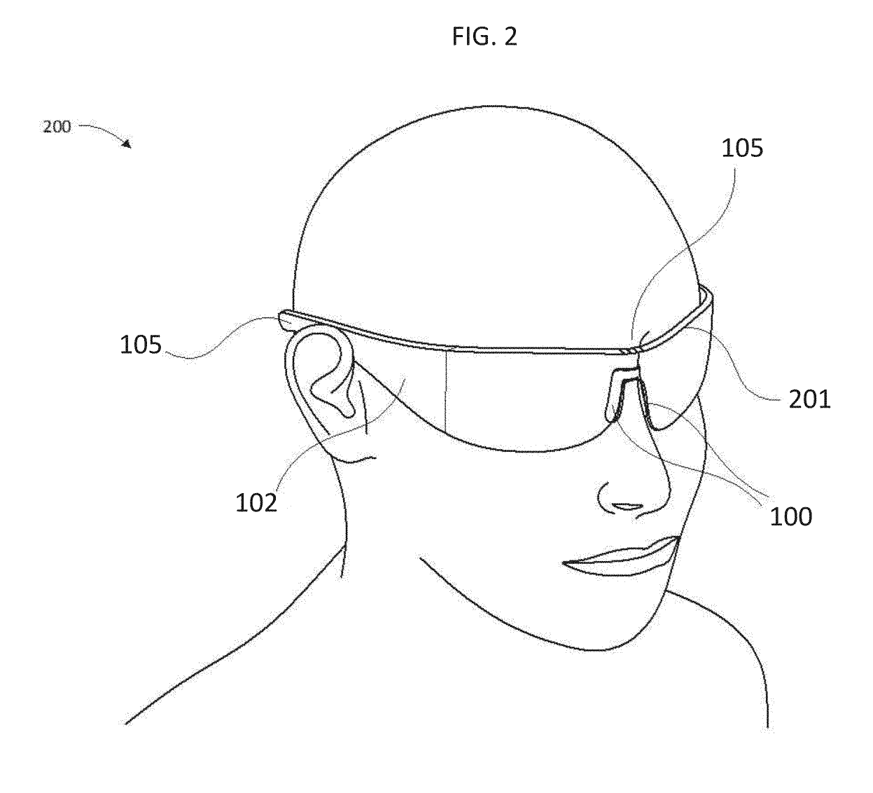

In reference to FIG. 2, the wearable bio-sensing device 200 is shown. In a specific embodiment, the device is a pair of bio-sensing glasses having a nose pad and a pair of temples. The device is meant to be worn as a normal pair of eyeglasses, with the nosepad resting on the nasal bone of a user and the temples contacting the temples of the user and resting atop each ear. The nose pad 100 is positioned as known in the art. Each nose pad 100 has one or more embedded sensors. In a preferred embodiment, the one or more embedded sensors detect pressure wave signals generated by the heart. The embedded sensors are in electrical communication with a control circuit 105. In an alternate embodiment, other sensors are in electrical communication with the control unit such as ALS (ambient light sensor) sensors and DMP (motion processor sensor) sensors. Each temple 102 of the glasses are comprised of a plurality of sensors, processors, electrodes, lights, circuits, memory units, and other transmitting components. These components will be further described herein.

In further reference to FIGS. 1 and 2, a bio-fluid electrode 104 is positioned on the temple and configured to collect, analyze, and transmit data regarding the presence of electrolytes, enzymes, and their levels. Each electrode may be adapted to collect, analyze, and transmit data regarding electrolytes and enzymes.

In further reference to FIGS. 1 and 2, each component is in electrical communication with a battery 103 with a charging component. An optional attachment port 101 is positioned on a terminal end of the temple 102. The optional attachment port 101 is able to engage with additional sensing components. A plurality of buttons, such as a home button 106 and mode button 107 allow the user to control the function and settings of the device. One or more LED's 109 are positioned on the device to alert the user to abnormal readings from the sensors, power status, charging status, among other visual cues known in the art.

In an embodiment, Nose Pad Construction includes sensing electrodes (100) signal-processing unit 105 and sensor control unit 111 performs computation on acquired data. It is responsible for feature extraction and anomaly detection. Nose Pad, 100 is in direct contact with Nose and rests on the Nasal Bone, and is responsible to acquire pressure wave signals generated by body, including heart. Signal processing tasks are performed on acquired signal, which include de-noising, decomposition, segmentation, and feature extraction. Pressure Wave Sensor extracts feature in time domain, frequency domain, cepstrum domain, and statistical domain. Enzyme and Electrolyte Sensor extract features in Time Domain only.

The bio-sensing glasses 200 are shown in an embodiment of the present invention. Specifically, the device comprises a pair of temples 102 and nose pads 100 positioned on a frame 201. The frame, nose pads and temples are a housing wherein the sensing elements are disposed.

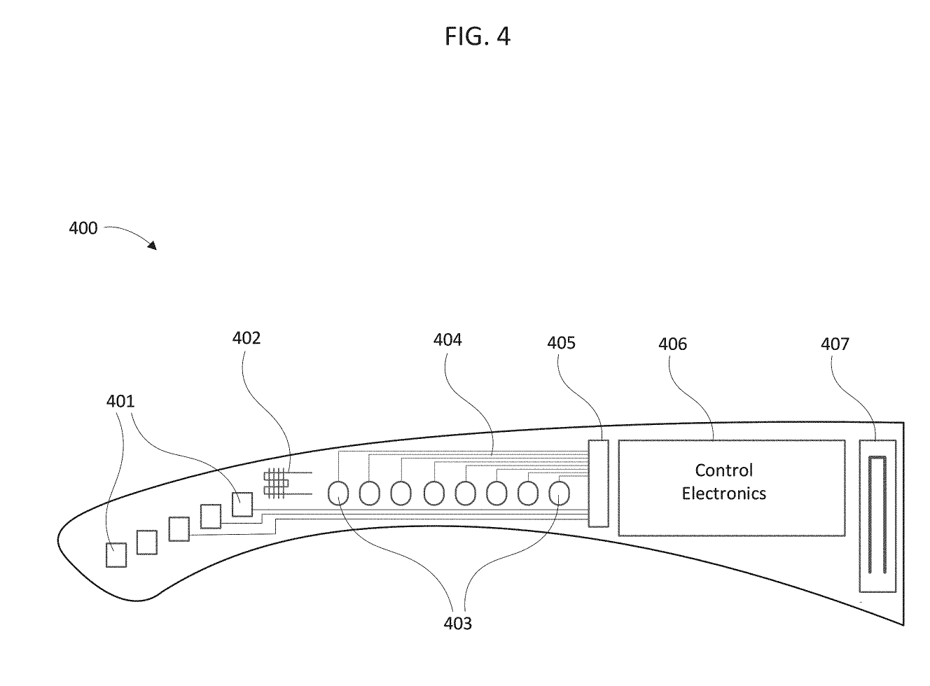

In reference to FIG. 4, the construction of the temple 102 includes bio-sensors, which are in direct contact with human skin, and are capable of computing enzyme and electrolyte level from human sweat. 401 are solid state electrolyte sensors 401 responsible to extract sodium and potassium Level from human sweat. 403 are solid state enzyme Sensors responsible to extract Glucose and Lactate Level from Sweat. (Sensors are sprinkled across the temple, with no specific fixed location). Temperature Compensation for Electrodes is provided by thermal sensor, 402.

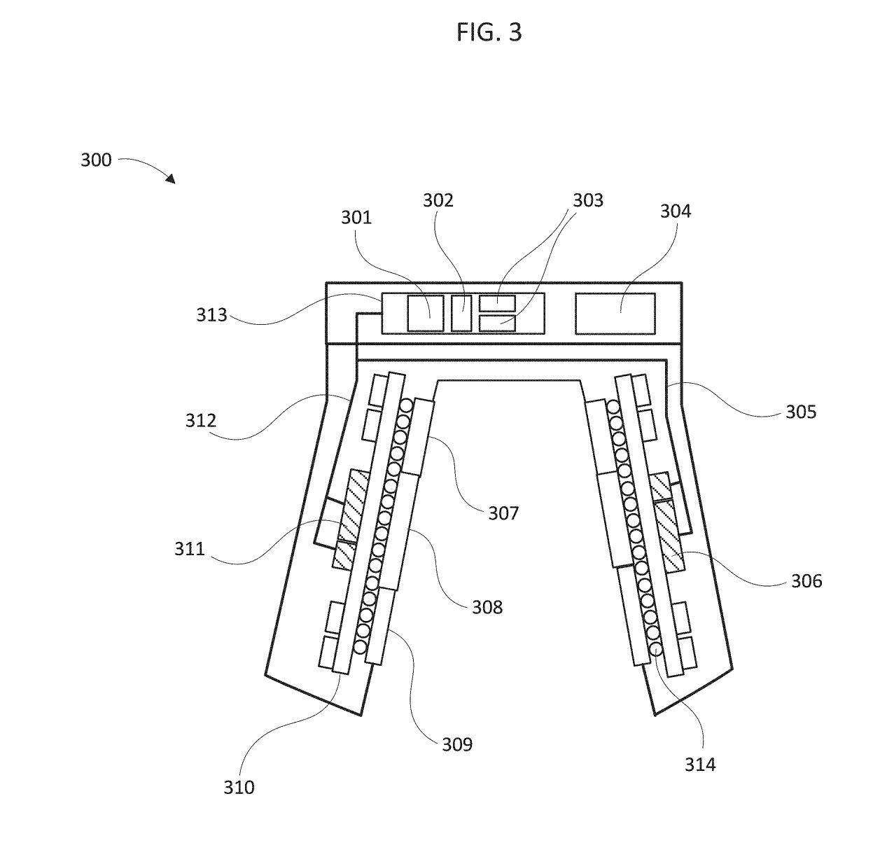

In reference to FIG. 3, the nose pad construction 300, includes 4 components listed herein;
(a) Left Pad Sensor Boards, 310, mounted with a nose pad sensor, 307, 308 and 309
(b) Right Pad Sensor Boards, 310, mounted with a nose pad sensor, 307, 308 and 309
(c) Control Board, 313, mounted with Signal Processor, 301, memory, 303 and power management device, 302 & interfaced to Right/Left sensor boards.
(d) Battery, 304

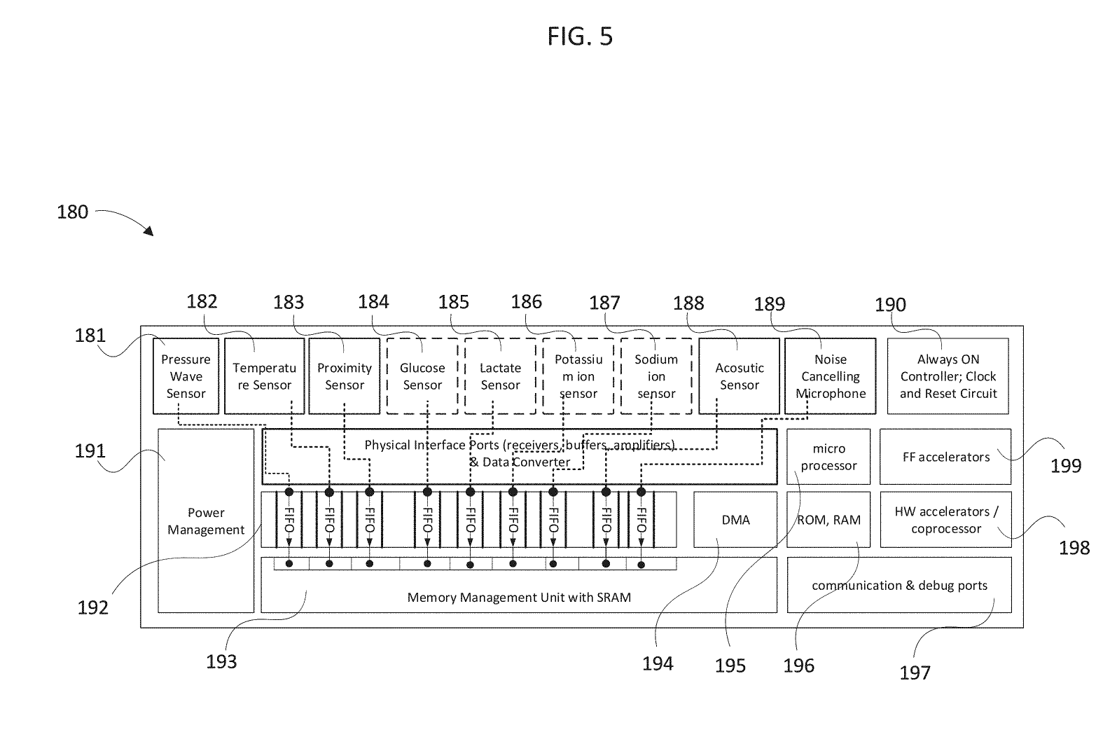

In reference to FIG. 5 which described nose pad sensor controller design, which provisions support for multiple sensors, 180, including proximity sensor, 183 to ensure device is in direct contact with body. In an embodiment, pressure Wave sensor 181, measure subtle changes/vibrations; temperature sensors 182 measure skin temperature; mems microphones capture acoustic signal, 188; along with noise cancelling microphone, 189.

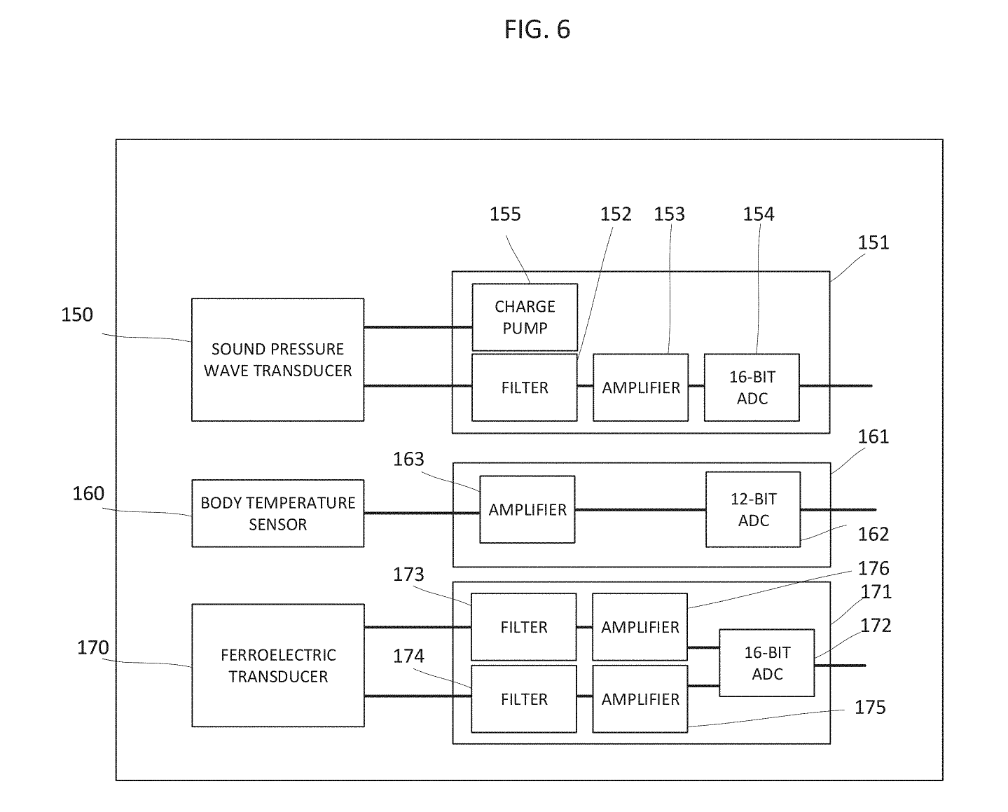

In further reference to FIG. 5, the nose pad package contains the above-mentioned sensors as well as an always-on ultra-low power microprocessor, 190 that performs sensor management and operations. A sensing silicon is positioned inside the nose pad, along with analog front end, data converter and memory as shown in FIG. 6. The microprocessor configures and controls the attached sensing elements. One or more fixed function accelerators perform de-noising, filtering and baseline wandering functionality. Hardware accelerators are configurable blocks performing pre-processing of acquired data. Sensors 181, 182, 183, 188 and 189 are present inside the Nose Pad. Construction of each sensor is discussed below.

In further reference to FIGS. 5 and 6, the Pressure Wave Sensor (181) is designed to capture and extract heart acoustic pressure waves when sensor is resting over the nasal bone, perform filtering and de-noising. This is accomplished by pre-processing the acquired signal, perform signal processing to analyze acquired data and detect if any structural problem, diseased vessels, or blood clot problems exists. Pressure Wave Sensor (181) Construction includes Sound Pressure Wave Transducer (150) and Ferro-electric Transducer (170) to capture pressure sound waves from the Nasal Bone. Analog Front End contains signal conditioning, power management for these sensors (151, 161, 171). Analog to Digital Converter provides digital output for each sensor. Acoustic Sensor (188) and Noise Cancelling Microphone (189) contains MEMS based sensor acquiring acoustic sound responsible to capture high frequency sound and ambient noise.

Pressure Wave Sensor (PWS) is an embedded component inside the nose pad, and it operation includes capture of pressure wave signals. Piezoelectric/Ferroelectric Thin Film Transducer, operating in low frequency range, to capture S3 and S4 components of heart sound wave.
a) Sensor Subsystem, provides controls to Pressure Wave Sensor (in addition it also provides control to Enzyme and Electrolyte Sensors). Ultra-Low Power Processor, is responsible to configure, control and operate the sensing devices.
b) Control Logic/State Machine provisions, control and operates Pressure Wave Sensor. Signal Conditioning Block which consists of Pre-Amplifier, Filter, and Programmable Gain Amplifier is responsible to remove noise and amplify the signal. ADC samples and provides digital equivalent of analog signal. Data is stored in memory which is used by hardware accelerator to extract cardiac cycle & store back into the memory.

Capturing pressure wave at the nasal bone, is achieved using ferroelectric/piezoelectric sensing electrodes, 308 and 309, along with sound pressure transducer (SPT), 307, to captures pressure wave signals generated by the body and also includes other environmental factors like breathing, speech, and more. Acquired pressure wave signal includes acoustic sound, primarily in infrasound and low audio frequency region, due to movement of heart muscles, blood flow, and opening and closing of heart valves.

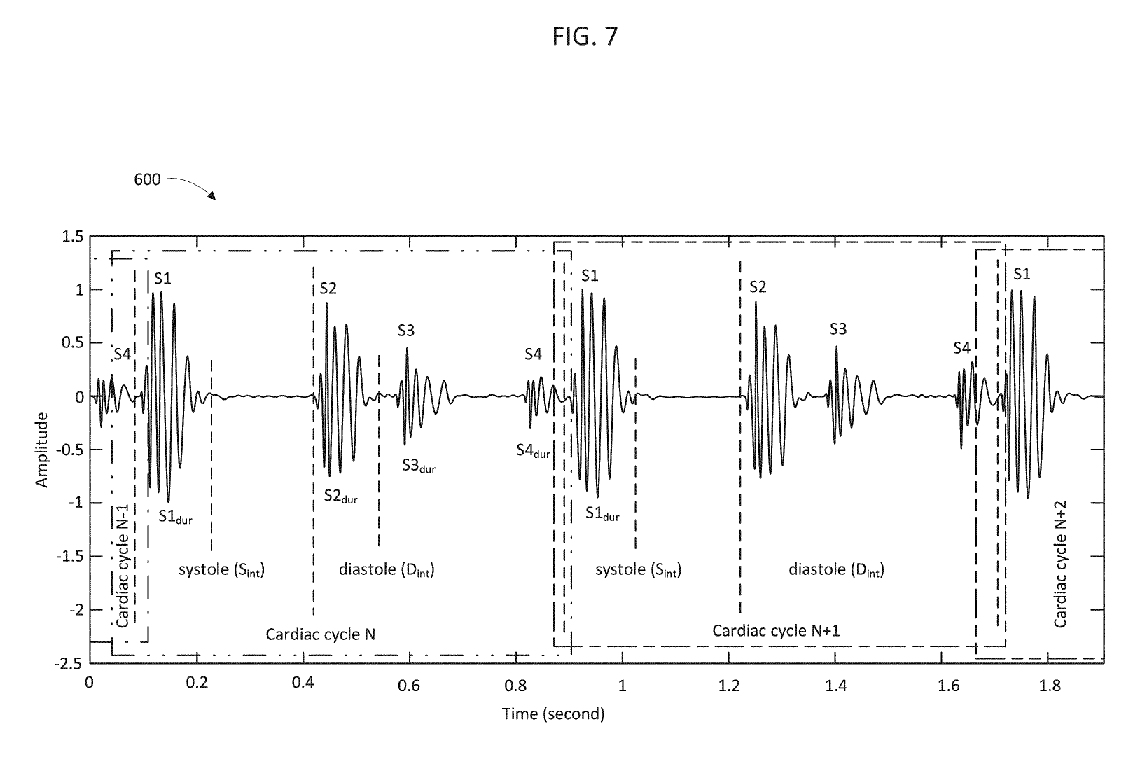

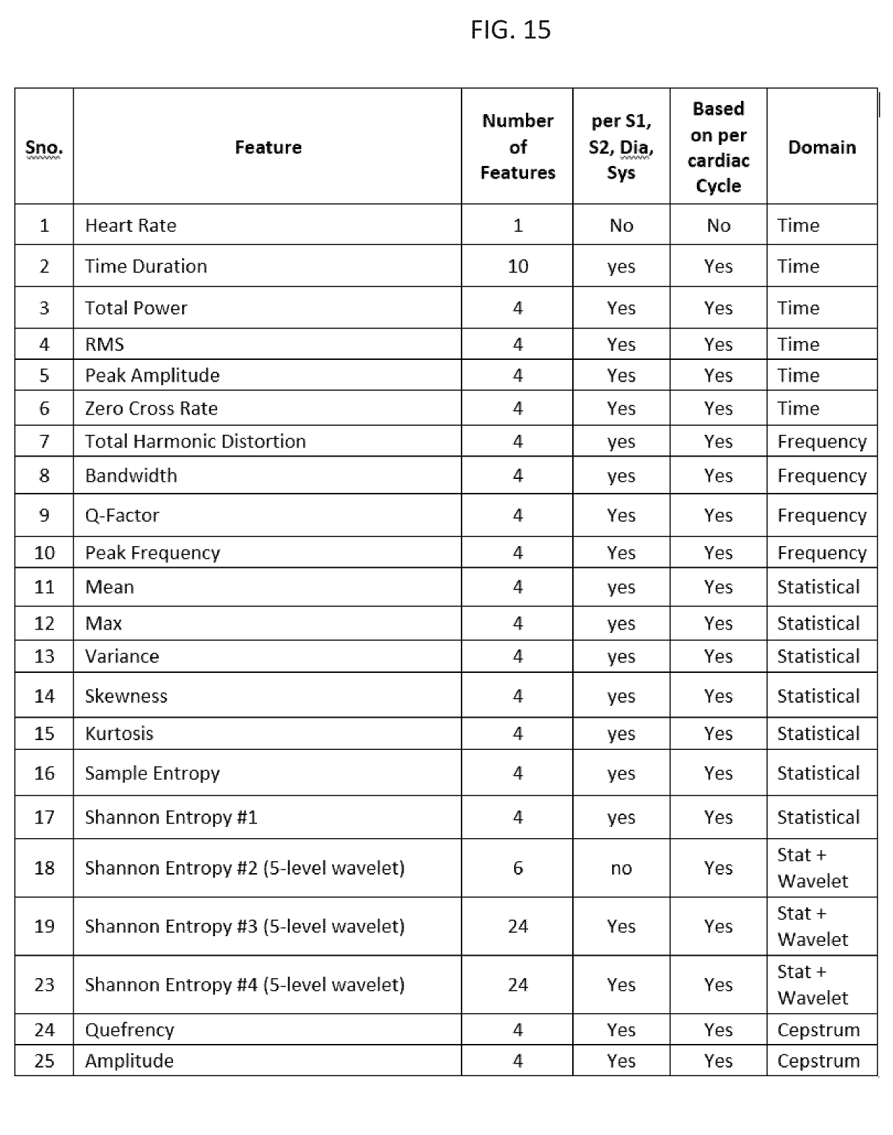

Construction of Nose Pad Module includes Digital Signal Processor, 301, Fixed Function Hardware Accelerators, 302, and memory blocks, 303, to perform numerous tasks on acquired data. These tasks are described below and referenced in FIG. 7 in an embodiment of the present invention.
a. Denoising and filtering, needs to be performed before Segmentation. Common noise sources include endogenous or ambient speech, motion artifacts, and physiological sounds such as intestinal and breathing sounds. Other physiological sounds of interest, such as murmurs, clicks, splitting of the heart sounds and additional S3 and S4 sounds can also complicate the identification of the S1 and S2 sounds. A de-noising fixed function accelerator performs the task of removal of ambient noise, power-line noise, sensor movement artifact, and baseline wandering, in addition to respiratory noise, bowel noise, and in-audible and audible sound generated by the body.
b. Cyclic detection fixed function accelerator performs the task of detecting the cyclic capture of heart sound wave. This is equivalent to providing a windowing effect to the acquired sound wave and separate each cycle's data from another. The cyclic detection fixed function accelerator provides markers for 51, S2, S3 and S4-, which contain frequency, amplitude and time information.
c. Segmentation is done by fixed function accelerator, and is based on the data provided by cyclic detection unit. Segmentation Block divides the waveform into small cardiac cycle waveforms, each containing information pertaining to one cycle only.
d. Segmentation is an essential step in the automatic analysis of heart sound recordings, allowing for analysis of the periods between these sounds for the presence of clicks and murmurs. The hardware accelerator is configured by loading the fixed operating kernel to perform specific tasks.
e. Feature Extraction on Segmented Data is done by Digital Signal Processor, 301. DSP computes the features in time, frequency and statistical domain, which is provided in the table of FIG. 15.

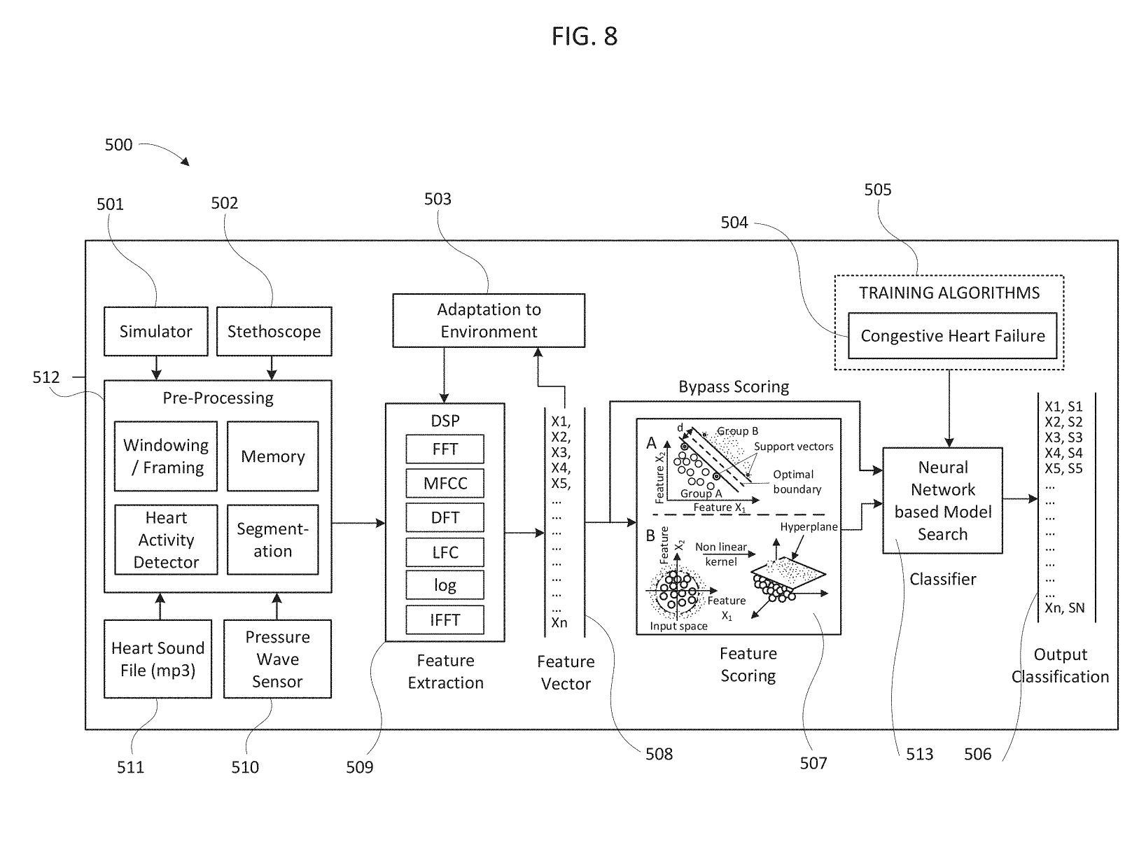

End to End Data Path is represented In FIG. 8, describing support for sensors/data source, 501, 502, 510, 511 Pre-Processing, 512, feature extraction from segmented heart sound signal, using Digital Signal Processor, 509, generation of feature vector, 508, to provide Output Classification, 506, based on Training Algorithms, 505, and Training Data Sets, 504.
(a)—Pressure Wave Sensor, 510, provides heart sound to pre-processing block, 512, which performs Filtering, Windowing, and Segmentation. This Data is stored into Local memory.
(b)—Captured Heart Sound, requires validation, which is done using Heart Activity Detector, which is located inside pre-processing block, 512. This is done to ensure that acquired heart sound is Valid, coming from an adult human. Heart Activity Detector, 890, also validates placement of sensing device, 863, and provides placement assistance to the consumers.
(c)—Heart Activity Detector, 890, is a Fixed Function Accelerator, performs various computational techniques to validate heart activity. based on cepstrum analysis and Energy Levels. This ensures that the sensor is able to compute valid heart activity based on data generated by the heart, by identifying quefrency components of S1, S2, S3, S4, Peak Power Level, Cyclic Rate and more. Computed Values are compared with minimum set threshold levels. Valid Heart Signal is indicated by a valid signal, 892.
(d)—Pre-processing Memory Block stores windowed and segmented data, along with Cardiac Cyclic Information, for direct consumption by Digital Signal processor, 509.

In further reference to FIG. 8, a signal processing unit, operates on acquired biological data set to extract features 509 set using various techniques such as; FFT, IFFT, DFT, LFC, CWT, and DWT. Feature vectors 508 are generated representing relationships between various extracted features for each data-set. Feature vectors 508 generated by the vector processor do not take into account any environmental condition, in-die and process variations, or thermal noise and need to be compensated for the same. Adaptation Block, 503, provides compensation against variables.

Feature scoring 507 provides a mechanism for a backend model search to better verify the outcome and assist in accurate computation based on training data. The feature scoring mechanism is introduced to generate rank-based system, enabling faster compute time by comparing against known good trained data set and signed data set.

Output classifications 506 on acquired data sets are done using Classifier, 513, which utilizes machine learning techniques such as; support-vector-machine (SVM), hidden Markov Model (HMM), linear/logistic regression, kNN, Naive Bayes classifier, Back-propagation, and Boltzmann machines.

Classification algorithms 505, used by Classifier, 513, is based on labeled data sets to accurately classify heart disease. Classification of abnormal heart signals are based on extracted feature sets from acoustic and electrical data sets regressed over multiple cardiac cycles. Abnormalities are referred as deviation from normal heart signal. The tool uses maximum-margin classifier to classify abnormal heart signals.

Model search ranking is used to classify aortic/mitral regurgitation, aortic/mitral/pulmonary stenosis, pulmonary hypertension, hypertrophic cardiomyopathy, restrictive cardiomyopathy, atrial/ventricular septal defect.

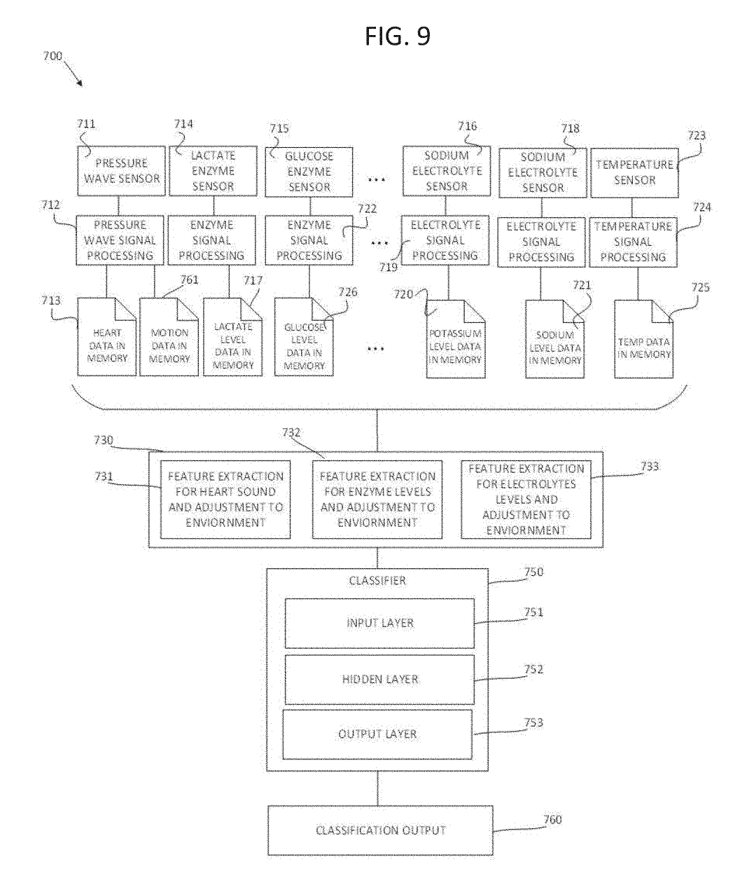

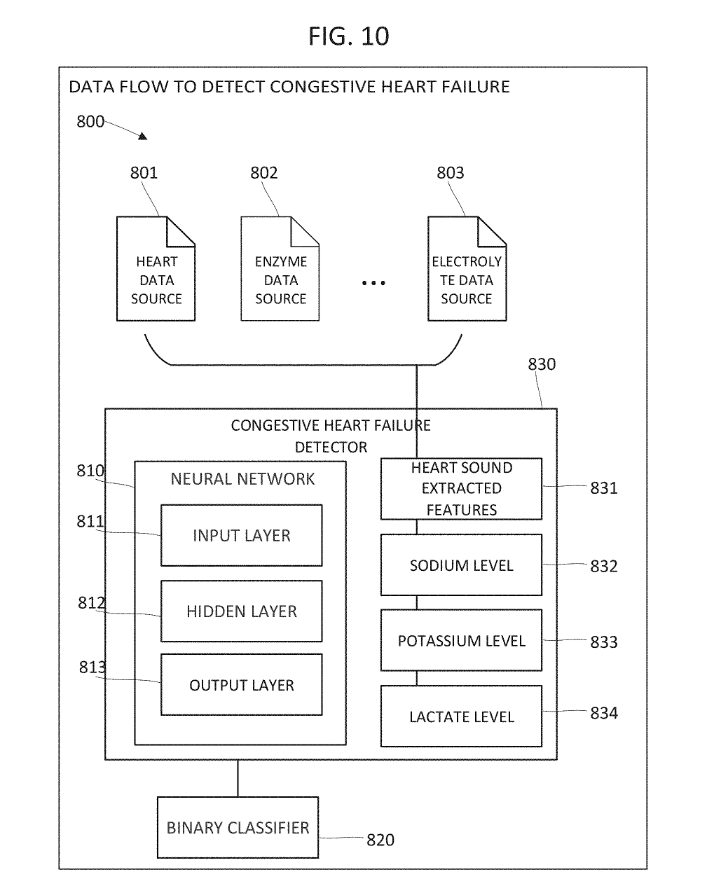

In reference to FIGS. 9 and 10, a process for classifying congestive heart failure is shown in an embodiment of the present invention.

Classification of Congestive Heart Failure is done using Nose Pad as Sensing Device along with Artificial Intelligence and Machine Learning Techniques.
a) Capture Heart Sound Data 801 from nasal bone, is used to extract Heart Health information. Temple Sensing Devices is used to capture Enzyme 802, and Electrolyte 803, levels from Human Sweat.
b) Criteria used to Classify Congestive Heart Failure is the
   i. Abnormal Heart Sound, and Presence of S3 component in captured heart sound
   ii. Elevated Level of Sodium Electrolyte. and
   iii. Dwarfed Level of Potassium Electrolyte and Lactate Enzyme.
c) Nose Pad Sensing Device senses and computes
   i. presence of S3 in acquired heart sound,
   ii. time period of S3,
   iii. Peak Amplitude and Quefrency of S3 in Cepstrum Domain,
   iv. Spectrum Analysis for presence of S3 in frequency domain, and
   v. Measure S2-S3 time duration (between 120 and 180 ms).
a. Temple Sensing Device senses and computes
   i. Sodium and Potassium Level in Human Sweat
   ii. Lactate and Glucose Level in Human Sweat
d) Signal Processor extract above and provide above mentioned features to a neural network, which classifies output, and provides binary classification.

In specific reference to FIG. 10, heart data, enzyme data, and electrolyte data sources 801, 802, 803, transmit data to the congestive heart failure detector 830. As described herein, data is analyzed and transformed until a binary classification is reached by the binary classifier 820.

Human sweat is rich in physiological information and is used to detect cardiovascular diseases by capturing and analyzing sweat for levels of sodium, potassium, lactate and glucose.

The human sweat sensing device provides vital information at the molecular level using integrated solid-state non-invasive sensing elements. The human sweat sensing device contains two different type of electrodes—enzyme sensing electrodes and electrolyte sensing electrodes. Both electrodes operate upon contact with human sweat and generated appropriate electrical signal indicating level of sodium, potassium, and glucose.

The enzyme biosensor performs bio-fluid sensing, by sampling human sweat generated by the human body. Sweat captured by the sensors is pre-processed using the signal conditioning block and analyzed using signal processor to detect glucose and lactate level present in sweat.

Electrolyte biosensor 716 and 718 performs bio-fluid sensing by sampling human sweat generated by the human body. Sweat is captured by the sensors 716 and 718 is processed using signal conditioning block and analyzed using signal processor 719, and computed electrolyte levels are stored in memory, 720. Abnormal level of electrolytes in sweat can disrupt the overall balance and functioning of the nerves, cardiovascular system, and muscles. Normal level of potassium is critical to the proper function of nerve and muscles cells, particularly heart muscle cells. Normal levels of sodium are critical to maintain normal blood pressure, supports the work of your nerves and muscles, and regulates your body's fluid balance.

Enzyme biosensor 714 and 715 performs bio-fluid sensing by sampling human sweat generated by the human body. Sweat is captured by the sensors 714 and 715, which is processed using Enzyme Signal Processor, 722, and computed enzyme levels are stored in memory, 717 and 726.

Sweat sensing devices are designed to be wearable sensor, and operates when in direct contact with the skin. This device is used for continuous monitoring of body vitals at the molecular level, and functions by capturing sweat generated by human body and by selectively screening for biomarkers in the sweat.

Both enzyme and electrolyte bio-sensors 714, 715, 716 and 718 are designed as a fully integrated solid-state biosensor, containing sensing electrodes, signal conditioning blocks, and signal processing blocks, along with power, clock and reset management.

The signal processing unit 719 and 722 embedded inside the sensor analyzes acquired electrolyte and enzymes signals, to detect abnormal levels, in order to maintain an overall balance and functioning of the nervous system, cardiovascular system, and muscles.

The analog front end, as described in FIG. 6, provides interface to enzyme and ion exchange based biosensor for signal conditioning (amplification, filtering, and data-converter) of sensor signals. Signal processor converts acquired signal from each sensor to sensor data which is used to classify abnormalities.

Biosensors are mounted on the inside of the temple in a wearable glass, which is in direct contact with the skin and sweat, and allows monitoring of glucose, lactate, sodium and potassium level from sweat easily, as larger quantity of sweat can be captured for analysis at the temple compared to the nose pad.

In addition to biosensors, other sensors like temperature sensors, proximity sensors, accelerometers, compasses, and gyroscopes are placed on the nose pad and on the temple. These sensors are used to correlate and validate biosensor data and detect physical activities by itself. For example: a user performing cardiovascular exercise, when perspiration starts, will have elevated heart rate, excessing blood flow, higher body temperature, higher lactate level, as well as higher sodium and potassium levels, however these will stabilize during cardiovascular exercise. The goal of the device is to detect that user is performing cardiovascular activity and report the vitals during exercise using these additional sensors.

Placement of sensors inside the temple is shown in FIG. 4. Enzyme and electrolyte biosensors, 401, 403 along with Temperature Sensor, 402, are placed on the inner side of the temple, making direct contact with the skin to capture sweat. Pre-processing of acquired enzyme and electrolyte signal is done using a digital signal processor, 406. A hinge, provides contact to the glasses and is also used for data interface to data-converter/signal processor.

All solid-state ion exchange Bio-sensors are electrochemical ion sensors converting the activity of a target ion into an electrical potential as the measurable signal. Silicon construction includes solid-state ion-selective electrodes and reference electrodes, which converts the activity of a specific ion dissolved in a sweat into an electrical potential.

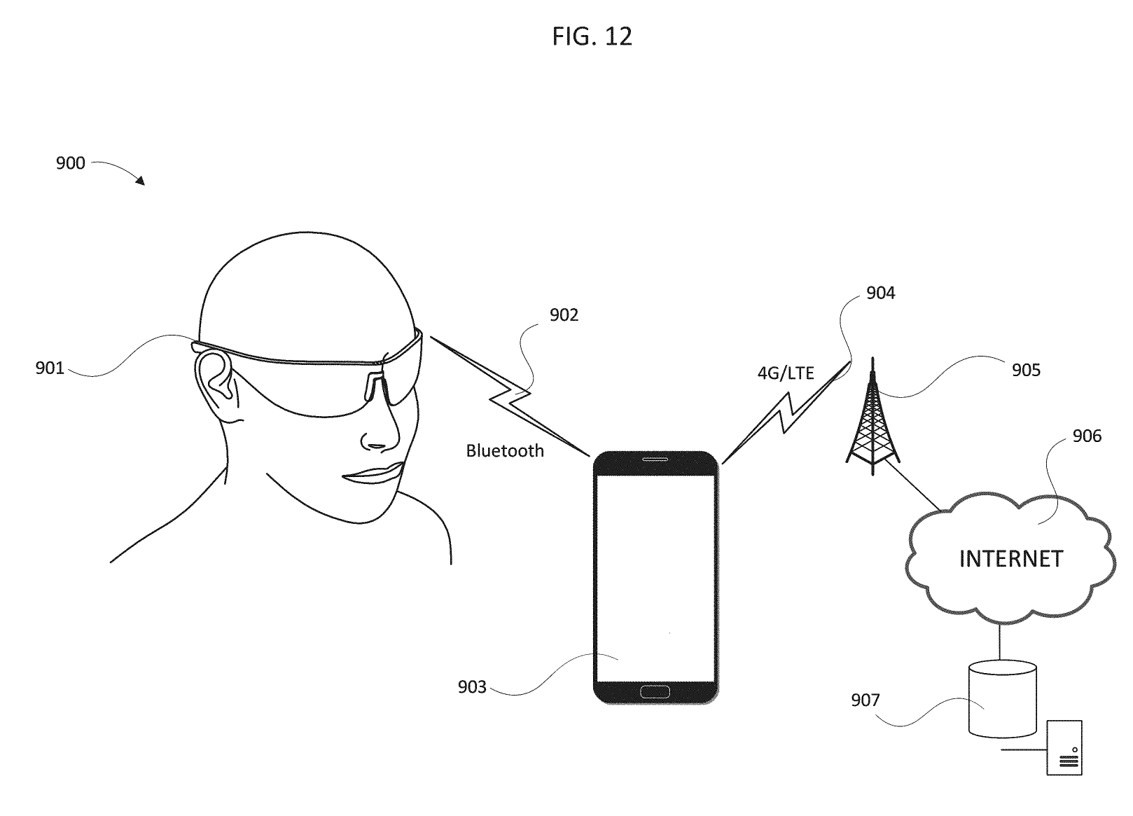

In reference to FIGS. 12, 13 and 14, wearable bio-sensing glass, 901 communicates with user's smart phone, 903 providing user interface for control, configuration, and operation of the device. This interface is provided using Bluetooth low energy (BLE), 902. BLE interface is used to transmit user's biological data acquired by bio-sensing glass. User control of the device is provided using a mobile application. This application provides health of the device, operating status, and controls to each component inside the device. The user will be able to monitor health and life of every sensor and measurement performance.

The mobile device application consists of a Graphical User Interface (GUI), providing controls for device operation. It also includes Bluetooth framework to establish connection with sensing device. The GUI includes a signal wave viewer to display acquired waveforms and playback heart sound using smart phone speakers (headphones) and Perform processing to detect any abnormality in acquired data. It can also upload data files to the cloud and download data files from the cloud.

The mobile device application provides the user with updated status of their heart health by reporting the following abnormal heart sound, murmur, heart rhythm disorder, heart rate, heart rate variability, regurgitation, stenosis, arterial hypertension, hypertrophic cardiomyopathy, restrictive cardiomyopathy, atrial septal defect, and ventricular septal defect along with sodium, potassium, and glucose levels.

The mobile device application provides mechanisms to map and label data during physical exercise and report selected vitals to the user.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A bio-sensing device comprising:
a. a pair of eye-glasses, having a pair of temples, and a nose pad; and
b. a plurality of sensors disposed within the pair of eye-glasses;
wherein the plurality of sensors are adapted to acquire, analyze, and classify biometric information.

2. The device of claim 1, wherein the biometric information is selected from a group consisting of; pressure, acoustics, temperature, glucose, lactate, potassium ions, and sodium ions sensing elements.

3. The device of claim 2, wherein an acoustics sensor is adapted to receive biometric acoustic signal comprising of heart sound to detect and classify murmur; rhythm disorder heart rate variability, stenosis, regurgitation.

4. The device of claim 1, wherein at least one of the plurality of sensors is one or more pressure sensors, wherein at least one of the pressure sensors receives a heart pressure wave signal, and wherein at least one of the pressure sensors receives lung sounds.

5. The device of claim 1, wherein at least two of the plurality of sensors are electrodes, and wherein the at least one electrode is an enzyme sensor, and at least one electrode is an electrolyte sensor, wherein each electrode receives information from human sweat.

6. The device of claim 5, wherein the information received from human sweat is selected from a group consisting of; glucose, lactate, potassium ions, and sodium ions.

7. The device of claim 1, wherein the at least one pressure wave sensor is positioned on the nose pad.

8. The device of claim 1, wherein the at least one enzyme sensor is positioned on at least one of the pair of temples.

9. The device of claim 1, wherein at least one pressure wave sensor is a piezoelectric sensor or a ferroelectric sensor, and wherein the piezoelectric sensor or ferroelectric sensor are adapted to receive heart sound.

10. The device of claim 1 further comprising sensors selected from a group consisting of; one or more accelerometers, one or more gyroscopes, one or more compasses, and one or more altimeters to determine user behavior and activity while the measurement is being recorded.

11. A method for monitoring congestive heart failure comprising the steps of:
a. capturing pressure waves using one or more pressure wave sensors, wherein at least one of the sensing device is a pressure wave sensor;
b. filtering pressure waves using one or more fixed function hardware accelerators;
c. collecting the heart pressure wave over an n number of cardiac cycles;
d. extracting one or more features from each of the n number of cardiac cycles using a digital signal processor, wherein the one or more features extracted in one or more time domain features, one or more frequency domain features, one or more statistical domain features, and one or more cepstrum domain features; and
e. providing extracted features to one or more classifiers, wherein each classifier is built using a neural network, wherein the neural network provides one or more output classifications, wherein the one or more classifications determine the presence of one or more abnormal heart sounds and behavior.

12. The method of claim 11, wherein abnormal heart sounds are classified by the presence of murmur or the presence of S3/S4 components, wherein the presence of murmur is classified as systolic murmur, diastolic murmur, or systolic-diastolic murmur.

13. The method of claim 11, wherein the step of one or more fixed function hardware accelerators filtering the pressure waves further comprises the steps of:
a. filtering pressure waves to isolate heart pressure waves; and
b. filtering one or more autoencoder reconstruct corrupted sections of heart sound.

14. The method of claim 11, wherein extracted features are functions based on one or more time domain features are defined, by total power and peak amplitude during the systolic period, total power and peak amplitude during the diastolic period, a zero crossing rate, time duration of one or more S1 heart sounds, time duration of one or more S2 heart sounds, time duration from the end of an S1 to the start of an S2 in one of the n number of cycles, and time duration of the end of an S2 to the start of an S1 in one of the n number of cycles, wherein the one or more frequency domain features are defined by one or more systolic periods, wherein the one or more statistical domain features are defined by mean, standard deviation, variance, skewness, kurtonsis, sample entropy, and shannon entropy, and wherein cepstrum domain features are defined by cepstrum S1 peak amplitude, S1 peak quefrency, cepstrum S2 peak amplitude, and S2 peak quefrency.

15. The method of claim 11, wherein filtering is facilitated by an environmental hardware accelerator, wherein the environmental hardware accelerator filters pressure waves from a user's environment.

16. The method of claim 11, wherein a plurality of networks classify the abnormal heart sound data, wherein a final output classification is provided by the one or more classifiers.

17. The method of claim 11, wherein algorithms selected from a group consisting of; Artificial Bee Colony and k-Nearest Neighbor are used to classify congestive heart failure, wherein congestive heart failure classification results are sub-classified as at risk, high risk, developed risk, and require intervention.

18. The method of claim 17, wherein the classification is reinforced using two or more electrode sensors.

19. The method of claim 18, wherein at least one electrode sensor is an enzyme sensor, wherein lactate levels reinforce the classification, wherein at least one electrode sensor is an electrolyte sensor, wherein sodium and potassium levels reinforce the classification.
